(19) United States
(12) Patent Application Publication
(10) Pub. No.: US 2014/0135611 A1
(43) Pub. Date: May 15, 2014

(54) ROBOTIZED SYSTEM FOR MOVING A REMOTELY GUIDED TOOL

(71) Applicant: Christophe Loustaudaudine, Areines (FR)

(72) Inventor: Christophe Loustaudaudine, Areines (FR)

(73) Assignee: ADVANCED ECHO TECHNOLOGY, Huisseau-en-Beauce (FR)

(21) Appl. No.: 14/130,567

(22) PCT Filed: Jun. 29, 2012
(86) PCT No.: PCT/IB2012/001290

§ 371 Date: Jan. 2, 2014

(30) Foreign Application Priority Data
Jul. 1, 2011 (FR) 11/02082

(51) Int. Cl. A61B 19/00; A61B 8/00

(52) U.S. Cl. CPC A61B 19/2203; A61B 8/4218; A61B 8/4444; A61B 8/461
USPC 600/407

(57) ABSTRACT

The subject of the invention is a robotized system of the type including a support onto which is placed an assembly of articulated arms at the end of which is disposed a tool in contact with a determined work surface, the system allowing the displacement of the tool in rotation about the point of contact of the tool with the work surface, in accordance with signals for controlling a module suitable for calculating them as a function of the remote handling of a virtual tool. The system is suitable for moving the point of contact of the tool in translation and this translation is controlled by the module in terms of polar coordinates, so as to have an angular displacement of constituent elements of this same support. The invention is more particularly useful in respect of tele-echography operations.

The present invention relates to a robotized system for moving a remotely guided tool.

The robotized system according to the invention applies notably to the practice of remote echographic examination, known as robotized remote echography, in which an echography examination is performed on an isolated site by virtue of a robot present on this site and remotely operated by a qualified doctor. The movements of the hand of the doctor, who controls a “joystick” forming a imaginary probe, are retransmitted to the real probe present at the end of the robot and applied to the patient in the isolated site. The robot reproduces the movements imparted by the doctor substantially in real time and the doctor receives the image picked up by the real probe on a control monitor.

A robot is known from the document FR 2 791 294 which comprises a plate with a shaft, a satellite holder mobile in rotation about the axis of the shaft, a second shaft mounted to rotate on the satellite holder, a tool holder mounted on the second shaft, the tool being mounted to rotate in the tool holder.

In its main embodiment, this robot is used for a movement with three components of rotation of an echographic probe about a fixed point which is embodied by the end of the probe in contact with the body of the patient.

There is also provided a support for the robot, with the support which makes it possible to perform translations in a horizontal plane passing through the fixed point. This translational movement is ensured by four toothed wheels arranged in pairs symmetrically around the support and the eccentricity of the mounting of which allows for a translation in a given direction and in its perpendicular direction to transcribe the translational movement of the imaginary probe into reproducible Cartesian coordinates.

In this context, the invention aims to improve a robotized system for the practice in particular of echographic examination, by proposing a robotized system suitable for following the rotational and translational movements of a imaginary probe directed by a qualified person.

To this end, the invention proposes a robotized system in which a support to which is attached a set of articulated arms at the end of which is arranged a tool suitable for being in contact with a determined work surface, and in which the tool can be moved in rotation about its point of contact with the work surface, in accordance with movement instructions originating from a driving module. The movement instructions are calculated according to the remote movement of a virtual driving tool of said robotized system and they are here noteworthy in that, on the one hand, the system is suitable for moving the point of contact of the tool with the work surface in translation and in that, on the other hand, this translation of the tool is controlled by the driving module by polar coordinates, embodied by an angular movement of constituent elements of the support followed by a radial movement of constituent elements of this same support.

Thus, it is possible to propose, according to the invention, a more compact robotized system, such that the scope of application of this system is widened. It will thus be more easily possible to apply this robotized system for remote echography apparatuses installed in space shuttles for example. The robotized system according to the invention requires less clearance for the movement of a tool than was necessary hitherto in the case of a translational movement according to Cartesian coordinates.

The translational movement desired for the probe is decoupled into rotational and translational movements of constituent elements of the support.

To this end, one embodiment according to the invention is such that the support comprises a deck carrying the end of the set of articulated arms which is mounted in translation, to produce radial movement, in rails securely attached to a deck suitable for rotating on itself by a determined angle, to produce the angular movement upon the desired translation of the probe.

According to different features that are found combined in an embodiment described hereinbelow, the deck carries an actuator which drives in rotation a pinion suitable for cooperating with a rack formed on one of the rails securely attached to the plate. This actuator is driven by a control signal originating from the control module to produce the radial movement.

The plate also comprises a hole arranged between the rails and through which passes a first rotation shaft mounted to pivot on the support and securely attached to the set of articulated arms, the hole having a diameter suitable for allowing said radial movement.

According to a particularly advantageous feature of the invention, the control module uses the rotation of the set of articulated arms carrying the tool to compensate for the induced rotation of the tool by its angular movement upon its polar translation.

Thus, the tool arranged on site at the end of the robot keeps, in its displacement, the same orientation as the virtual tool manipulated by the doctor. If this virtual tool is made to rotate the robotized tool is also made to rotate, whereas, if this virtual tool is only made to translate, without being rotated, an angle of rotation of the robotized tool is controlled which compensates for the angular rotation that is made necessary due to the transcription of the tool translation into polar coordinates.

According to one feature of the invention, the constituent elements of the support also comprise a fixed base of a portal structure inside which the plate rotates, said fixed base carrying a driving shaft which is mounted to rotate on said base, which is securely attached in rotation to the plate and which is associated with an actuator, said actuator being driven by a control signal originating from the control module to produce said angular movement.

One thus uses an axis of rotation of the set of articulated arms, already used elsewhere, for compensating for the rotation of the tool induced by the translation in polar coordinates.

According to one feature of the invention, the set of articulated arms comprises at least one top arm which is securely attached in rotation to a first shaft of the system mounted to pivot on the support, the control module generating a rotation of said top arm to compensate said induced rotation of the tool, wherein the axis of rotation of the top arm is parallel to the axis of the angular movement of the support. The set of articulated arms further comprises an intermediate arm securely attached to a second rotation shaft pivotedly mounted at the free end of said top arm, said intermediate arm bearing a third rotation shaft of which is securely attached in rotation a tool-holder arm on which the tool is mounted, such that three axes of rotation, defined respectively by the first shaft, the second shaft and the third shaft of rotation, are intersecting each other at the end of the tool. The control module is suitable for supplying, on the one hand, control signals to actuators associated respectively with each of the articulated arms to drive the rotation of the articulated arms about each of the three axes in order to control the rotational movement of the tool and supply, on the other hand, a reverse signal complementing the signal corresponding to the angular movement of the top arm to compensate the rotation of the tool induced by the angular movement of said support for the translational movement of the tool.

According to one feature of the invention, each actuator is associated with an angular position sensor suitable for determining the absolute angular position of the arm, of the shaft or of the pinion driven by said actuator.

The invention also relates to the application of such a robotized system to a remote echography device in which the movement of an echography probe is remotely produced, the remote echography device comprising, on the one hand, equipment on an appraisal site where a medical expert can remotely direct the echography via a virtual probe suitable for transmitting information to the control module of the robotized system in order to drive the movement of the tool, and, on the other hand, equipment installed in an operating site where the patient to be examined is located, and in which the operating site comprises a robotized system suitable for performing the echography on the patient according to the control information deriving from the movement of the virtual probe on the appraisal site.

Other features and advantages of the invention will emerge from reading the following description of one of its embodiments, illustrated by:

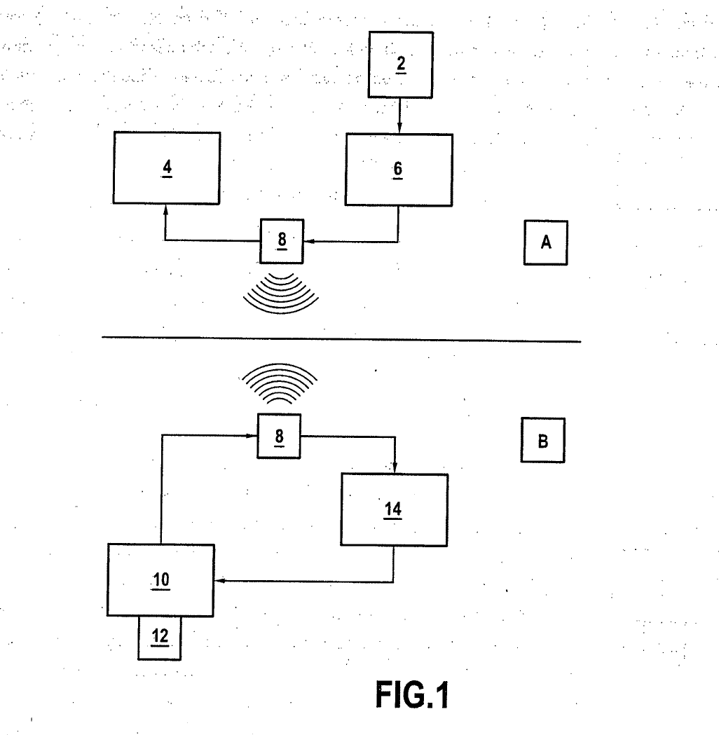

FIG. 1 which is a schematic illustration of a remote echography principle including the robotized system according to the invention in a particular embodiment;

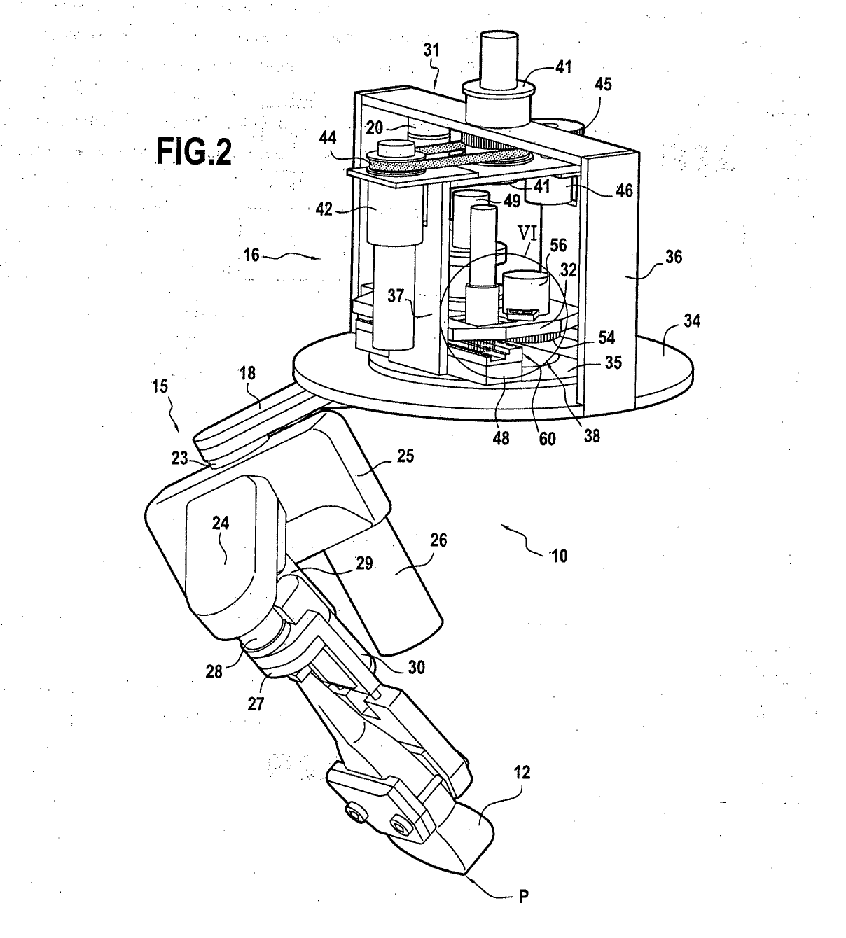

FIG. 2 is a perspective view of a robotized system according to the invention in the context of the application to a remote echography system illustrated in FIG. 1,

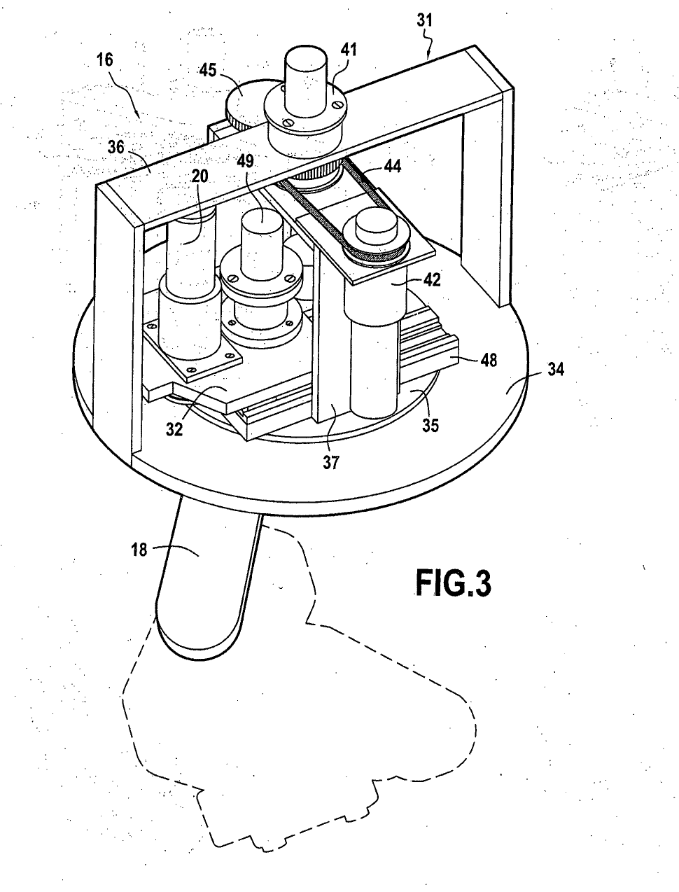

FIG. 3 is a perspective view, from above, of the system illustrated in FIG. 2,

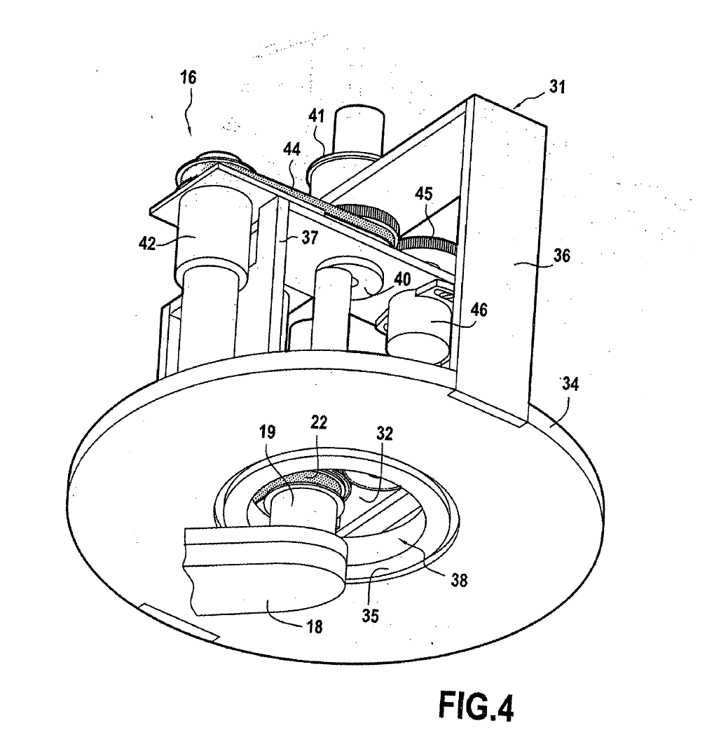

FIG. 4 which is an enlargement of the translational guiding device of the system of FIG. 2, with the more detailed representation of the portal structure and of the deck, seen from below,

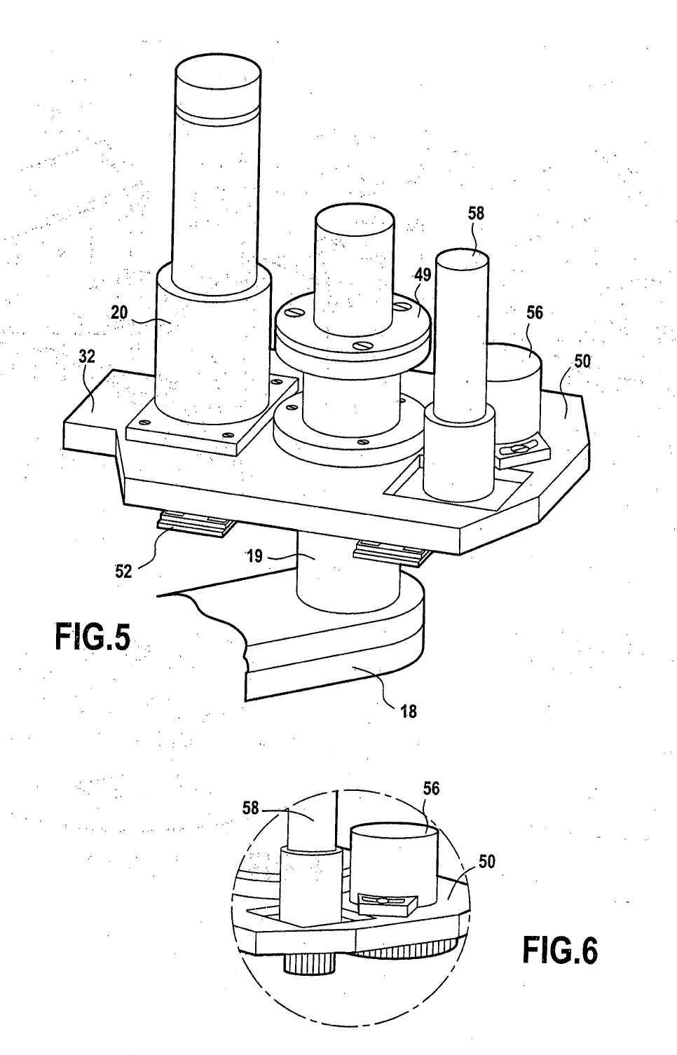

FIG. 5 is a partial view of the enlargement of FIG. 3, without the portal structure,

and FIG. 6 which is an enlargement of a detail of FIG. 2.

In the description that follows, without in any way limiting the scope, the context of the preferred application of the invention will be assumed, unless otherwise specified, that is to say the case of a remote echography robot, driven remotely by a health professional.

A remote echography device is represented in FIG. 1. It comprises, on the one hand, equipment on a site A where a medical expert can remotely direct the echography and, on the other hand, equipment installed in a site B where the patient to be examined is located.

The appraisal site A comprises a imaginary probe 2 that the medical expert can handle at will according to echographic image feedback from the patient which is given to the medical expert on the monitor 4. The movements of the imaginary probe 2 are measured by sensors integrated in this probe and the measured values are transmitted to a processing station 6, which codes these values in a computer frame so as to be able to communicate them via transceivers 8 from the appraisal site A to the operating site B.

The operating site B comprises a robotized system 10 suitable for performing the echography on the patient according to the control information deriving from the movement of the imaginary probe on the appraisal site A.

A real probe 12, arranged at the end of the robotized system, makes it possible to obtain echographic images which are sent via the transceivers, this time from the operating site B to the appraisal site A, to the monitor 4 so that the medical expert can view these images and adapt the movement on the imaginary probe to collect other images.

A control module 14 is suitable for driving the robotized system to reproduce the movements of the imaginary probe 2. To this end, the control module receives, via the transceivers 8, the coded values representative of the movement of the imaginary probe.

As illustrated in FIG. 2, the robotized system 10 comprises a set of articulated arms 15 carrying, at one end, the real echography probe 12, this set being mounted on a support 16. The set 15 and the support 16 are suitable for being driven according to control signals originating from the control station 14, which can be seen in FIG. 1. It is observed that, for practical reasons for reading FIGS. 2 to 6, the cables allowing for the transmission of the control signals, like the power supply cables, have not been represented.

The set of articulated arms 15 comprises a top arm 18 securely attached in rotation to a first shaft 19 of the system mounted to pivot on the support (and particularly visible in FIG. 5). This top arm comprises a first part which extends under the support radially to the first shaft and a second part which prolongs, in the radial direction of the arm, this first part opposite the first shaft, by forming therewith a determined angle.

A first actuator 20 is linked to this first shaft by a belt 22 (notably visible in FIG. 4) to drive it in rotation and to thus control the rotation of the top arm, of which the free end of the second part comprises a second rotation shaft 23 mounted to pivot and securely attached to an intermediate arm 24.

This intermediate arm is associated with a second actuator 26 and a belt (not visible here because it is hidden by a housing 25) which drive the rotation of the intermediate arm relative to the top arm.

A tool-holder arm 27 is mounted securely attached in rotation to a third rotation shaft borne by the intermediate arm and it is associated with a third actuator 29.

The intermediate arm and the tool-holder arm are respectively associated with an absolute angular position sensor, in order to ensure the correct position of the arms relative to one another for the orientation of the probe at the end of the set of articulated arms to be correct.

The real probe 12 is mounted to rotate on this tool-holder arm with a degree of freedom in translation driven by a fourth actuator 30, in order to be able, on the one hand, to press the probe against the body of the patient and check that the point P is always in contact therewith regardless of the inclination of the probe to obtain good images and, on the other hand, slightly disengage the probe from the body of the patient if necessary to produce the inclination of the real probe.

The three rotation shafts described above rotate about competing axes at a fixed point P corresponding to the end of the real probe 12, adapted to be in contact with the work area.

The intermediate arm has a form substantially equivalent to that of the top arm, namely a first part at right angle to the second rotation shaft 23 and a second part that is inclined. The spread and the angle formed between the two parts of the intermediate arm are determined for the probe to be able to take a position, here vertical, in the direction of the axis formed by the first shaft, that is to say with a first rotation shaft and a third rotation shaft which are aligned. As will be described later, the rotational movement of the probe about this fixed point is obtained by the rotational control of each of the arms relative to one another and relative to the support. During an echographic examination, the fixed point P of the real probe corresponds to the point of contact of this probe with the body of the patient here forming a work area over which the probe is adapted to be moved.

The support 16 can be seen in all the FIGS. 2 to 4, and details of this support can be seen in FIGS. 5 and 6. It comprises a portal structure 31 inside which a deck 32 is slidably mounted, which carries, among other things, the first shaft 19 of the set of articulated arms 15.

The portal structure 31 comprises, on the one hand, a fixed base 34, of annular form and topped by a first arch 36, and, on the other hand, a concentric annular plate 35 adapted to rotate inside the base in the plane defined by the latter and topped by a second arch 37. The plate 35 has an outer diameter substantially equal to the inner diameter of the ring forming the fixed base, and the second arch 37 is of lesser height than the first arch 36, so that the plate 35 can rotate inside the base with the second arch which passes under the first arch. The plate 35 is pierced at its center by a hole 38 of determined diameter, and it will be understood hereinbelow that the value of this diameter allows for a more or less lengthy translational clearance of the robotized system.

A driving shaft 40 (notably visible in FIG. 4) is securely attached to the plate 35 via the second arch 37 and it is coupled to a rotating slip ring 41 attached to the first arch 36 securely attached to the base 34 and the advantages for use of which will be described hereinbelow. This base remains fixed and the plate rotates within this base after the driving shaft 40 has been made to rotate by the action of a fifth actuator 42 fixed to the second arch 37 and belt 44 which links a pulley securely attached to the fifth actuator and a pulley arranged on the circumference of the rotating slip ring 41 securely attached to the first arch.

This rotating slip ring 41 comprises a fixed part securely attached to the first arch and whose circumference comprises, in addition to the pulley for the belt drive, a toothed belt adapted to mesh with a pinion 45 mounted on the second arch and associated with an angular position sensor 46 which determines the absolute position of the plate. The rotating slip ring also comprises a part (not visible in the figures) that moves rotationally inside the fixed part and which is securely attached in rotation to the driving shaft 40 and the second arch 37.

As will be described hereinbelow, the plate 35 is adapted to rotate by a determined angle, and the deck 32 is mounted in such a way as to follow this rotation, in this way rotationally driving the set of articulated arms. In each of the angular positions of the plate, the deck can be driven in translation relative to this plate, in this way driving the set of articulated arms in translation.

Two parallel rails 48 are fixed for this purpose to the plate 35, on either side of the hole 38, being adapted to receive the deck.

This deck 32 takes the form of a substantially planar plate 50, which carries, substantially at its center, a rotating slip ring 49 associated with the first shaft 19. The deck 32 is adapted to slide in the rails 48 via slides 52 attached under the plate 50. Here, there are two slides per rail.

As described previously, the first actuator 20 is adapted via the associated belt 22 to drive the first shaft 19 in rotation relative to the deck. The latter also comprises a pinion 54 which is mounted under the deck and which is adapted to cooperate with a toothed belt extending around the first shaft 19 to enable the angular position of the first axis to be controlled. This pinion 54 is associated with an angular position sensor 56 which determines the absolute angular position of the first shaft 19 of the set of articulated arms.

The deck also comprises a sixth actuator 58 which controls the rotation of a meshing pinion 59 (visible in FIG. 6 and represented by dotted lines in FIG. 2) and which is arranged under the plate to cooperate with a straight line of teeth forming a rack on an outer edge 60 along a rail 48. This way, as will be described later, the deck can slide relative to the plate, in the axis of the rails.

There now follows a description of the specific case of a translational movement of the real probe and of the point of contact at its end, following a translational movement remotely controlled by the medical expert. To this end, the control module generates, on the one hand, an angular movement signal, that it transmits to the fifth actuator 42, and, on the other hand, a radial movement signal, that it transmits to the sixth actuator 58 associated with the deck of the portal structure.

The fifth actuator 42 is controlled to drive in rotation, via the belt 44, the driving shaft securely attached to the plate. The plate thus pivots relative to the base of the portal structure, which generates a rotation of the set of articulated arms and a different orientation of the probe.

It is observed that, on the expert site A, the virtual probe has been moved in translation by the medical expert while retaining the same inclination and without any rotation. To reproduce this final result of a pure translation, with no rotary component, the control module generates, defacto, a complementary control signal for the first actuator 20 to start rotating the top arm of the robot, in order to compensate the rotation of the probe induced in the angular movement of the deck and thus correctly orient the probe head relative to the orientation of the imaginary probe maneuvered by the medical expert.

It can be seen in FIGS. 2 to 4 that the top arm is arranged in such a way that its axis of rotation is parallel to the axis of the driving shaft 40. The rotation of the probe induced by the angular movement of the deck is a rotation about the axis of rotation of the plate supporting the deck, that is to say about the axis defined by the driving shaft 40. An induced rotation by an angle value α will then be compensated by a rotation of the top arm by a reverse angle value −α about its axis of rotation defined by the first shaft 19 and parallel to the driving shaft 40.

At this stage, the deck has been pivoted by an angle α corresponding to the angular coordinate of the desired translational movement and this deck, carrying the set of articulated arms and the probe, has to be moved in translation by the radial coordinate determined by the control module.

To this end, the sixth actuator 58 is driven to drive the associated pinion in rotation, said pinion being engaged with the rack securely attached to one of the rails. The pinion-rack meshing enables the deck to move along the rails. The hole 38 through which the first shaft 19 of the system passes allows the radial movement of this shaft following the movement of the deck. The hole has a determined diameter corresponding to the authorized translational clearance of the probe. It is then important, during the design of the robot, to find a trade-off between the desire to have a sufficient translational clearance of the first shaft in the hole and therefore of the probe on the body of the patient, and the desire to have a compact robotized system.

The result of this is that the first shaft 19, and therefore the set of articulated arms, has been translated in the plane of the deck by an angular movement and a radial movement. During this translation, the probe head has remained oriented correctly relative to the orientation of the imaginary probe, by virtue of the compensation controlled by the module 14.

It will be understood from the above that, for a translation of the probe along the body of the patient to be controlled, the robotized system according to the invention offers the advantage of using control signals relating to polar coordinates, notably in the interests of robot compactness. The induced rotation of the probe in the angular movement of the support specific to the desired translational movement of this probe is managed and compensated by the design of the robot and at least by the arrangement of the top arm and of the first shaft directly attached to the support, which makes it possible to compensate this induced rotation, whereas it does not exist when considering using Cartesian control signals as is known.

There now follows a description of the use of the robotized system according to the invention in echographic examinations.

On the operating site, the robot is positioned in such a way that the end of the real echography probe 12 is in contact with the work area, here the body of the patient, facing the area to be examined.

The echographic images taken by the probe are sent via the transceivers to the monitor present on the appraisal site and these images are analyzed by the medical expert.

In order to perfect his or her analysis, the medical expert seeks to obtain other images, either by tilting the probe relative to its point of contact, or by moving this point of contact. To do this, the medical expert performs corresponding movements on the imaginary probe so that said movements are reproduced on the operating site via the robotized device.

The rotational and translational movements of the imaginary probe are translated and transmitted to the control module via the processing station and the transceivers as has been described previously by the creation of a byte-based computer frame representative of these movements. The control module then determines what should be the movements of the support and of each of the arms of the set of articulated arms to faithfully reproduce the movement of the probe at the end of the robotized system, and it generates corresponding control signals for each of the six actuators of the robotized system, that is to say for each of the four actuators associated with the set of articulated arms and for each of the two actuators associated with the support. It will be understood that the computation matrices incorporated in the control module and adapted to transform the desired movement of the probe into control signals for the arms of the robotized system are known and that they could take different values to be adapted to different types of robot and notably to particular arrangements of the arms.

Advantageously, in order to be able to manage in real time a possible movement of the probe simultaneously in translation and in rotation, the control module permanently calculates instructions for all the actuators of the robotized system.

The movements made by the medical expert on the imaginary driving probe are thus remotely reproduced by the real probe which is moved over the body of the patient and enables echographic images to be taken.

In real time, the control module receives, on the one hand, the instructions for moving the real probe, to transmit them in as many control signals as the particular case requires and it receives, on the other hand, information originating from the actuators and from the angular position sensors so as to have reliable real time data concerning the positioning of each of the arms and of the support, in order to be able to calculate the appropriate control signals subsequently to drive the robot in accordance with the instructions from the medical expert.

If the medical expert wants only an inclination of the probe about the fixed point embodied by the contact of this probe with the patient by retaining the position of this fixed point, the control module sends a control signal to each of the actuators arranged in the set of articulated arms, that is to say to the first actuator associated with the top arm, to the second actuator associated with the intermediate arm and to the third actuator associated with the tool-holder arm, as well as to the fourth actuator for the translation of the probe along the axis of the tool-holder arm, against the body of the patient. At the same time, the control signals corresponding to the fifth and sixth actuators remain unchanged, no translation of the set of arms being desired.

If the medical expert wants only a translation of the fixed point while keeping the same inclination, the control module simultaneously generates an angular movement signal, that it transmits to the fifth actuator, a radial movement signal, that it transmits to the sixth actuator, as well as, as has been described previously, a complementary control signal for the first actuator, the reverse of the angular movement signal transmitted to the fifth actuator, for the first actuator to compensate the rotation of the probe in the angular movement of the deck. At the same time, the control signals corresponding to the second, third, and fourth actuators remain unchanged.

It will be observed that, in both cases, the first actuator is always driven. If the medical expert wants to move the point of contact of the probe and at the same time modify the inclination of the probe about this fixed point, new control signals are sent simultaneously to the six actuators and the control signal sent to the first actuator corresponds to the addition of the two control signals provided respectively for the inclination in the rotation of the probe and for the angular compensation in the translation of the probe. It will be understood that the addition of a control signal and of a complementary control signal is intended here to mean an addition or a subtraction of the absolute values of these signals, to take account of the angle values, possibly negative, relative to a given position of the top arm.

On reading the above, it will easily be observed that the invention clearly achieves the aims that it sets out to achieve and to recall them all would be pointless. Thus, a robotized system is proposed which allows a specialist doctor to perform an echography and establish a reliable diagnosis on a remote patient. The medical issue is how to extend the movements of the hand of the doctor to a dedicated robot and, in the present case, make it possible to follow the adjustment movements in translation that the doctor wants to make for greater visibility. This effective tracking of the movement of the doctor is performed with a compact system enable it to be used in any conditions and in any location. The use of a polar control and the associated structure of the support of the robot make it possible to reduce the footprint of the system while keeping the same functionalities and in particular a given translational clearance.

The position of the probe is consequently reproduced in real time, with a position of the point of contact which observes the translational movement of the hand of the doctor, an inclination of the probe relative to the work area and this point of contact which is also observed, and an orientation of the probe which is not affected by the fact that the translation is taken into account, by virtue of the angular compensation which is applied in real time to the rotational control of the top arm, about an axis parallel to the axis of rotation of a rotating element of the support.

The presence of the rotating slip rings makes it possible to dissociate the electrical cables attached to the fixed part and those attached to the rotating part. It is consequently possible to rotate without distinction in one direction of rotation or in the other without worrying about a maximum number of turns in one and the same direction to be observed to avoid breaking the cables and breaking the electrical contact. As has been described previously, for practical reasons in reading the figures, the cables have not been represented, but it will be understood that they are associated with the fixed and rotating parts of the rotating slip rings in a manner known elsewhere. Here, the use of rotating slip rings is advantageous in that the control module has no additional mathematical constraints to determine whether such or such a part of the slip ring must be rotated in such or such a direction. The rotating slip ring 49 associated with the first shaft 19 is particularly advantageous in that it makes it possible to render the two levels that are the support and the set of articulated arms independent. The control module can rotate the arms in a direction without worrying about the direction in which the plate of the support will rotate.

In variants that are not detailed on the drawings, it will be possible to provide for the following, without the list being exhaustive:
the robot carrying the real probe is implemented otherwise provided that it is associated with a support allowing its translational movement according to polar coordinates; herein, the robot should have a top arm mounted to rotate about an axis parallel to the axis of rotation corresponding to the angular displacement control for the support in the context of its translation by polar coordinates;
by way of example, the real probe 12 can be mounted on the tool-holder arm without being driven by a dedicated actuator as has been described previously with the fourth actuator, but by rather forming the object of a passive assembly with a return spring system tending to bring the end of the probe into contact with the work area. It will be understood that, without departing from the context of the invention, the control module generates signals suited to each situation seen previously for five actuators instead of six;
the angular position sensors associated with each of the arms of the robot can be relative sensors set on initialization. It will be understood that, in the embodiment described, the benefit of using absolute position sensors is that they make it possible to avoid a calibration step;
the appraisal site does not include a control station and the control module arranged on the operating site receives directly, via the transceivers, the values measured by the sensors incorporated in the imaginary probe;
the operating site also comprises an echographic monitor and this monitor sends as output video images via the transceivers to the monitor on which the medical expert relies for his or her examination;
the medical expert can give his or her instructions remotely by different types of imaginary tools. While it is particularly advantageous for the expert to be able to handle a single probe capable of transmitting information concerning the desired translational and rotational control, provision can be made, for a simplified design, for the medical expert to handle a imaginary probe to determine the inclination to be imparted and for him or her to simultaneously handle a joystick-like control to drive the translation of the probe.

Thus, the invention is not limited just to the device conforming to the embodiment explicitly described in light of FIGS. 1 to 6, and it should also be noted that the invention is not limited to the preferred application relating to remote echography operations. It can also be used for the manipulation of tools in machining conditions which require a separate control and command center, for example in hostile environments.

1. A robotized system comprising a support to which is attached a set of articulated arms at an end of which is arranged a tool suitable for contacting a determined work surface, said system being so designed as to move the tool in rotation about a point of contact of said tool with said work surface in accordance with movement instructions originating from a control module suitable for calculating such instructions as a function of a remote movement of a virtual driving tool of said robotized system, wherein said system is also suitable for moving said point of contact of the tool in translation and wherein such translation is controlled by said control module in polar coordinates, said translation of said point of contact of the tool being thereby produced through an angular movement of constituent elements of said support followed by a radial movement of constituent elements of same support.

2. The robotized system as claimed in claim 1, wherein said constituent elements of said support comprise a plate suitable for rotating on itself to produce said angular movement as well as a deck which carries the end of said set of articulated arms and which is mounted in translation in rails securely attached to said plate to produce said radial movement.

3. The robotized system as claimed in claim 2, wherein said deck carries an actuator driving in rotation a pinion which is suitable for cooperating with a rack formed on one of said rails secured to the plate, said actuator being driven by a control signal originating from said control module to produce said radial movement.

4. The robotized system as claimed in claim 3, wherein said plate comprises a hole which is arranged between said rails and through which passes a first rotation shaft pivotedly mounted on said support and securely attached to said set of articulated arms, said hole having a diameter suitable for allowing said radial movement.

5. The robotized system as claimed in claim 1, wherein said constituent elements of said support further comprise a fixed based of a portal structure inside which said plate rotates, said fixed base carrying a driving shaft which is rotatively mounted on said base, which is securely attached in rotation to said plate, and which is associated with an actuator, said actuator being driven by a control signal originating from said control module to produce said angular movement.

6. The robotized system as claimed in claim 1, wherein said control module generates a rotation control signal for at least one arm of said set of articulated arms carrying the tool, said control signal being suited for compensating for a rotation of the tool induced by its angular movement during polar translation.

7. The robotized system as claimed in claim 6, wherein said set of articulated arms comprises at least one top arm which is securely attached in rotation to a first shaft of said system pivotedly mounted on said support, said control module generating a rotation of said top arm compensating for said induced rotation of the tool, wherein the axis of rotation of the top arm is parallel to the axis of the angular movement of the support.

8. The robotized system as claimed in claim 7, wherein said first shaft is mounted on said support coupled to a rotating slip ring securely attached to said support.

9. The robotized system as claimed in claim 7, wherein said set of articulated arms further comprises an intermediate arm securely attached to a second rotation shaft pivotedly mounted at the free end of said top arm, said intermediate arm carrying a third rotation shaft to which there is securely attached in rotation a tool-holder arm on which the tool is mounted, and wherein three axes of rotation are defined respectively by the first shaft, the second shaft, and the third shaft, said three axes intersecting each other at said tool end, wherein said control module is adapted to supply, on the one hand, control signals to actuators associated respectively with each of the articulated arms to drive the rotation of the articulated arms about each of the three axes in order to control the rotational movement of the tool, and, on the other hand, to supply to the actuator associated with the top arm, an additional signal reverse to the signal corresponding to the angular movement, to compensate for the rotation of the tool induced by the angular movement of said support due to the translational movement of the tool.

10. The robotized system as claimed in claim 9, wherein each of said actuators is associated with an angular position sensor adapted to determine the absolute angular position of said arm, said shaft, or said pinion driven by said actuator.

11. The application of the robotized system as claimed in claim 1 in a remote echography device for remotedly producing the movement of an echography probe wherein said remote echography device comprises, on the one hand, equipment on an appraisal site where a medical expert can remotely direct the echography and, on the other hand, equipment installed in an operating site where a patient to be examined is located, and wherein the operating site comprises a robotized system adapted for performing the echography on the patient according to said control information derived from the movement of a virtual probe in the appraisal site.

12. The robotized system according to claim 9, wherein:
said constituent elements of said support comprise a plate suitable for rotating on itself to produce said angular movement as well as a deck which carries the end of said set of articulated arms and which is mounted in translation in rails securely attached to the plate to produce said radial movement, said deck carrying an actuator driving in rotation a pinion which is suitable for cooperating with a rack formed on one of said rails secured to the plate, said actuator being driven by a control signal originating from said control module to produce said radial movement;
said constituent elements of said support comprise a fixed base of a portal structure inside which said plate rotates, said fixed base carrying a driving shaft which is rotatively mounted on said base, which is securely attached in rotation to said plate, and which is associated with an actuator, said actuator being driven by a control signal originating from the control module to produce said angular movement; and
each of said actuators is associated with an angular position sensor adapted to determine the absolute angular position of said arm, said shaft, or said pinion driven by said actuator.
